(19) United States
(12) Patent Application Publication
(10) Pub. No.: US 2010/0261251 A1
(43) Pub. Date: Oct. 14, 2010

(54) MICROBIAL KINETIC RESOLUTION OF ETHYL-3,4-EPOXYBUTYRATE

(75) Inventor: Won Jae Choi, Singapore (SG)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Agency for Science, Technology and Research, Singapore (SG)

(21) Appl. No.: 12/668,659

(22) PCT Filed: Jul. 12, 2007
(86) PCT No.: PCT/SG2007/000208

§ 371 Date: Jun. 22, 2010

(51) Int. Cl. C12N 9/14; C12N 1/20; C12P 41/00

(52) U.S. Cl. 435/195; 435/252.1; 435/280

(57) ABSTRACT

Two novel microorganisms, Acinetobacter baumanni ATCC PTA-8303 and Cryptoeoecus albiduz ATCC PTA 8302 having epoxide hydrolase activity are described. The epoxide hydrolases from these microorganisms can be used to selectively hydrolyze (via epoxide opening) one enantiomer of ethyl-3,4-epoxybutyrate in a racemic mixture by a kinetic resolution process to result in the accumulation of the other enantiomer. Methods of preparing the microorganisms and their use in a kinetic resolution process of racemic ethyl-3,4-epoxybutyrate are also disclosed.

### BACKGROUND OF THE INVENTION

Enantiopure chiral compounds play an important role in the chemical and pharmaceutical industry. Regulatory requirements, the prospects for lower toxicity and higher efficacy of pharmaceutical compounds have increased the demand for enantiopure compounds. In response to this trend, there is growing interest in developing new chemical and biological production methods for preparing chiral intermediates or “building blocks” in the commodity chemical industry to serve this growing demand. For example, chiral epoxides, which have broad applications in the chemical industry (e.g., for the synthesis of pharmaceuticals, agrochemicals, as well as many fine chemicals), have a high market demand for their production. A major challenge in organic chemistry synthesis is to generate such compounds in high yields, with high stereo- and regio-selectivities.

Enantiopure chiral epoxides can be used as key intermediates in the preparation of more complex optically pure bioactive compounds for industrial purposes. Specifically, the chiral epoxide, ethyl 3,4-epoxybutyrate (EEB), comprises a terminal epoxide with ethyl ester group and in its enantiopure form (i.e., (R)-EEB or (S)-EEB), is an indispensable key intermediate in the synthesis of chiral pharmaceuticals and fine chemicals. EEB is a versatile synthetic intermediate as it undergoes stereospecific ring-opening reactions to form bifunctional compounds.

(R)-EEB is directly converted to (R)-δ-amino-β-hydroxybutyric acid ((R)-GABOB), neuromediator with antiepileptic and antihypertensive activity (see, Tetrahedron, 46, 4277 (1990)), and to (R)-carnitine (vitamin Bt), an aperitive agent for treating congestive heart failure or arrhythmia that has essential role in fat metabolism and widespread applicable as dietary supplement and antiobesity agent (see, J. Org. Chem., 53, 104 (1988), U.S. Pat. Nos. 4,865,771, 5,248,601, and 6,342,034). (R)-EEB is utilized as a C4 chiral synthon for the synthesis of ethyl (R)-4-chloro-3-hydroxybutyrate ((R)-CHBE) (see, Tetrahedron Lett, 33, 1211 (1992), J. Org. Chem., 49, 3707 (1984)); and (R)-4-hydroxy-2-pyrrolidone (see, Synthesis, 614 (1978)). (R)-EEB is also useful intermediates for the synthesis of salicylate enamide anticancer agents, i.e., Lobatamide C (see, J. Am. Chem. Soc., 125, 7889 (2003)) and new nitrogen mustards, which are modified carnitine analogues (Bioorg. Med. Chem., 11, 325 (2003)).

Moreover, (S)-EEB can be efficiently converted to (R)-4-cyano-3-hydroxybutyric acid ethyl ester, which can be used to prepare the HMG-CoA reductase inhibitor, Atorvastain (LIPITOR®), a cholesterol lowering blockbuster drug (see, Tetrahedron Lett, 33, 2279 (1992)). (S)-EEB is also useful chiral intermediate for the synthesis of (S)-4-hydroxy-2-oxo-1-pyrrolidine acetamide (Oxiracetam®), a cardiovascular drug for use as a brain function enhancer or for dementia, such as Alzheimer (see, WO 93/06826). (S)-EEB can be applied to the synthesis of (S)-3-hydroxytetrahydrofuran, which is intermediate for AIDS drug, Agenerase® (see, J. Am. Chem. Soc., 117, 1181 (1995)). Furthermore, valuable C4 chiral synthons, such as (S)-3-hydroxy-δ-butyrolactone (U.S. Pat. No. 6,221,639) and (S)-3-hydroxy-4-bromobutyric acid (U.S. Pat. No. 6,713,290) can also be synthesized from (S)-EEB.

In view of the above, it is clearly apparent that EEB is a valuable synthetic intermediate, and that a cost effective manner to produce enantiopure EEB on an industrial scale is highly desirable.

There have been several approaches reported to prepare enantiopure EEB either by chemical or biological routes. In one instance, (R)- or (S)-EEB was synthesized by reacting 3,4-dihydroxybutyronitrile with a sulfonyl chloride, followed by reaction with alcohol in the presence of acid and accompanied by base-catalyzed cyclization (see, U.S. Pat. No. 5,079,382). However, this approach is not commercially viable as it requires the use of an enantiopure starting material that is not commercially available and expensive to produce.

In another approach, (R)-3,4-epoxybutyric acid and the salt thereof, is prepared by subjecting (S)-3-activated-hydroxybutyrolactone to a ring-opening reaction to obtain 4-hydroxy-3-activated hydroxybutyric acid, which cyclizes to form the epoxide and results in the inversion of the chiral center (see, U.S. Pat. Nos. 6,232,478, 6,342,034). However, due to low purity of commercially available (S)-3-hydrorxybutyrolactone (the synthesis of pure (S)-3-hydrorxybutyrolactone is not known), this approach produces the desired (R)-3,4-epoxybutyric acid product accompanied with many impurities and thus is not suitable for many commercial applications.

In another approach, racemic EBB was resolved into (R)-EEB by hydrolytic kinetic resolution catalyzed by chiral (salen) CoIII complex (see, J. Am. Chem. Soc., 124, 1307 (2002), U.S. Pat. No. 6,693,206). This process afforded both recovered unreacted epoxide and 1,2-diol product in highly enantioenriched form (i.e., >99% ee, 45.6% yield for (R)-EEB). However, the reaction process described in this approach requires additional handling procedures as the reaction process is initially exothermic and the reaction mixture be kept at a low temperature (i.e., 0° C.). Also, this approach is not applicable to the preparation of (S)-EEB.

In biocatalytic approaches, purified esterases have been applied to kinetic resolution of racemic 3,4-epoxybutyric acid alkyl esters. For example, iso- and n-butyl (R)-3,4-epoxybutyrate were prepared using commercially available enzyme, steapsin, (isolated from swine pancreas) with 95% ee and in about 33-38% yield (see, J. Org. Chem., 53, 104 (1988), U.S. Pat. Nos. 4,865,771, 5,248,601, EP 237983 A2). In another example, pig liver esterase was used for enantioselective hydrolysis of racemic methyl 3,4-epoxybutyrate to provide (R)-epoxide with moderate enantiomeric ratio value (E=11) (see, Tetrahedron Lett., 30, 2513 (1989)). However, these enzymatic kinetic resolution processes may not be applicable for industrial processes, as they require the use of high amount of expensive commercial steapsin (J. Org. Chem., 53, 104 (1988), U.S. Pat. Nos. 4,865,771 and 5,248,601, EP 237983A2) or esterase (Tetrahedron Lett., 30, 2513 (1989)) both derived from mammalian sources. Moreover, in the case for pig liver esterase moderate enantioselectivity and low reaction rate is observed.

The use of microbial epoxide hydrolases (EHs) as biocatalysts for the preparation of chiral epoxides and vicinal diols via the kinetic resolution of racemic epoxides has recently been recognized. As shown in FIG. 1, the selective hydrolysis of a racemic epoxide can generate both the corresponding diols and the unreacted epoxides with high enantiomeric excess (ee) values. Some success has been found for the use of microbial EHs to prepare epoxides and diols with good enantioselectivity in small-scaled syntheses (see, Current Opinion in Biotechnology, 12, 552 (2001); Current Opinion in Chemical Biology, 5, 112 (2001)). However, before a broad industrial platform for EH catalyzed resolution of racemic epoxides can be realized, several limitations to this approach need to be addressed.

First, the number of EH enzymes available for this transformation is still small and those that have shown promise in synthetic applications are even more rare (see, U.S. Pat. Nos. 5,849,568 and 6,387,668). In particular, the current discovery of new EHs through screening available strains is hampered by limited culture collections and the lack of powerful screening assays. Second, many known EH enzymes have limited substrate scope. In fact, among the available enzymes, many have selectivity for only one enantiomer and thereby does not provide access to both enantiomers for a particular compound. Finally, in many EH catalyzed resolution processes, high concentrations of enzymes (either whole cells or crude extract) and rather low substrate concentrations are required due to low catalytic efficiency for the enzyme.

In view of the above, there remains a need in the art for biocatalysts that could be utilized for the kinetic resolution of racemic epoxides to allow synthetic access to enantioenriched epoxides (and vincinal diols), in particular, for the enantioenriched epoxide EEB. It is desirable that the kinetic resolution process utilizing biocatalysts be both cost-effective and applicable to an industrial scale process. The present invention fulfills this and other needs.

### BRIEF SUMMARY OF THE INVENTION

In one aspect, the present invention provides for a biologically pure culture of the microorganism Acinetobacter baumannii characterized by the identifying characteristics of ATCC No.: PTA-8303, and derivatives and mutants thereof.

In another aspect, the present invention provides for a process for resolving a racemic mixture of ethyl-3,4-epoxybutyrate comprising culturing the microorganism Acinetobacter baumannii ATCC PTA-8303, which is characterized by its ability to preferentially hydrolyze (S)-ethyl-3,4-epoxybutyrate to form (S)-ethyl-3,4-dihydroxybutanoate, in the presence of the racemic mixture in an aqueous nutrient medium under aerobic conditions.

In yet another aspect, the present invention provides for a process for preparing (R)-ethyl-3,4-epoxybutyrate, the process comprising: (i) incubating a racemic mixture of ethyl-3,4-epoxybutyrate with an epoxide hydrolase in a form selected from the group consisting of whole cells, cell extracts, partially purified enzyme, purified enzyme and a mixture thereof, wherein the enzyme and the cell comprising the enzyme is from the microorganism Acinetobacter baumannii ATCC PTA-8303 and is characterized by its ability to preferentially hydrolyze (S)-ethyl-3,4-epoxybutyrate in the mixture to form (S)-ethyl-3,4-dihydroxybutanoate; and (ii) isolating (R)-ethyl-3,4-epoxybutyrate from the reaction medium.

In another aspect, the present invention provides for a biologically pure culture of the microorganism Cryptococcus albidus characterized by the identifying characteristics of ATCC No.: PTA-8302, and derivatives and mutants thereof.

In another aspect, the present invention provides for a process for resolving a racemic mixture of ethyl-3,4-epoxybutyrate comprising culturing the microorganism Cryptococcus albidus ATCC PTA-8302, which is characterized by its ability to preferentially hydrolyze (R)-ethyl-3,4-epoxybutyrate to form (R)-ethyl-3,4-dihydroxybutanoate, in the presence of the racemic mixture in an aqueous nutrient medium under aerobic conditions.

In another aspect, the present invention provides for a process for preparing (S)-ethyl-3,4-epoxybutyrate comprising the steps of: (i) incubating a racemic mixture of ethyl-3,4-epoxybutyrate with an epoxide hydrolase in a form selected from the group consisting of whole cells, cell extracts, partially purified enzyme, purified enzyme and mixtures thereof, wherein the enzyme and the cell comprising the enzyme is from the microorganism Cryptococcus albidus ATCC PTA-8302 and is characterized by its ability to preferentially hydrolyze (R)-ethyl-3,4-epoxybutyrate in the mixture to form (R)-ethyl-3,4-dihydroxybutanoate; and (ii) isolating (S)-ethyl-3,4-epoxybutyrate from the reaction medium.

In another aspect, the present invention provides for a method for producing the microbial epoxide hydrolase comprising the steps of: (i) aerobically cultivating the microorganism Acinetobacter baumannii ATCC PTA-8303 under conditions suitable for the formation of the epoxide hydrolase in a nutrient medium containing assimilable sources of carbon, nitrogen, and inorganic minerals; (ii) incubating the culture for a time sufficient to produce the epoxide hydrolase; and (iii) recovering the epoxide hydrolase from the nutrient medium.

In another aspect, the present invention provides for a method for producing the microbial epoxide hydrolase comprising the steps of (i) aerobically cultivating the microorganism Cryptococcus albidus ATCC PTA-8302 under conditions suitable for the formation of the epoxide hydrolase in a nutrient medium containing assimilable sources of carbon, nitrogen, and inorganic minerals; (ii) incubating the culture for a time sufficient to produce the epoxide hydrolase; and (iii) recovering the epoxide hydrolase from the nutrient medium.

In another aspect, the present invention provides for the use of an epoxide hydrolase in a form selected from the group consisting of whole cells, cell extracts, partially purified enzyme, purified enzyme, and a mixture thereof, wherein the enzyme is derived from the microorganism Acinetobacter baumannii ATCC PTA-8303 for preparing (R)-ethyl-3,4-epoxybutyrate from a racemic mixture of ethyl-3,4-epoxybutyrate.

In another aspect, the present invention provides for the use of an epoxide hydrolase in a form selected from the group consisting of whole cells, cell extracts, partially purified enzyme, purified enzyme, and a mixture thereof, wherein the enzyme is derived from the microorganism Crytococcus albidus ATCC PTA-8302 for preparing (S)-ethyl-3,4-epoxybutyrate from a racemic mixture of ethyl-3,4-epoxybutyrate.

Other objects, features, and advantages of the present invention will be apparent to one of skill in the art from the following detailed description and figures.

### BRIEF DESCRIPTION OF THE DRAWINGS

FIG. 1 show a synthetic scheme which illustrates an enantioselective hydrolysis of a racemic epoxide with an epoxide hydrolase.

FIG. 2 shows an microscope image of the microorganism Acinetobacter baumanni ATCC PTA-8303.

FIG. 3 illustrates a synthesis of rac-EEB.

FIG. 4 illustrates the research strategy utilized in the discovery of a microorganisms of the invention.

FIG. 5 shows a graph which illustrates the concentration of (R)- and (S)-EEB present in a reaction mixture as a function of time during the kinetic resolution process rac-EEB catalyzed by EH from Acinetobacter baumanni ATCC PTA-8303.

FIG. 6A and FIG. 6B shows a synthesis scheme of alkyl-3,4-epoxybutyrates.

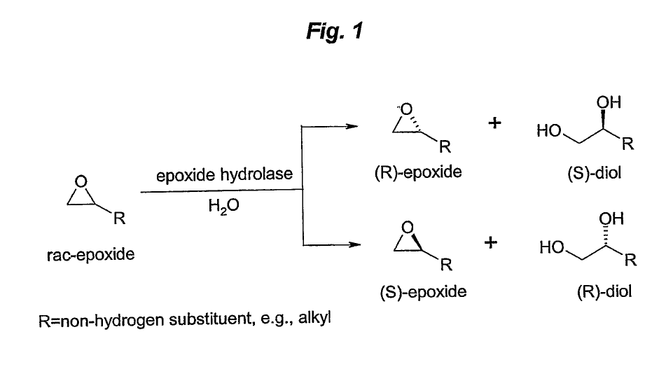

### DETAILED DESCRIPTION OF THE INVENTION

### Definitions and Abbreviations

As used herein, the term “enantioselectivity,” as measured by the “E” value, includes the selective capacity of an enzyme to generate from a racemic substrate, one enantiomer relative to the other in a product racemic mixture; in other words the E value is a measure of the ability of the enzyme to distinguish between enantiomers. A non-selective reaction has an E value of 1, while resolutions with E values above 20 are generally considered useful in synthesis. The enantioselectivity resides in a difference in conversion rates between enantiomers. Reaction products are obtained that are enriched in one of the enantiomers; conversely, remaining substrates are enriched in the other enantiomer. For practical purposes it is generally desirable for one of the enantiomers to be obtained in large excess.

The calculation of E is based on the measurement of two of three variables: (1) enantiomeric purity of the starting material (eeₛ); (2) enantiomeric purity of the product (eeₚ); (3) and the extent of conversion (c). Then one of the three equations can be used (see, Hydrolases in Organic Synthesis, Bomscheuer, U. T. and Kazlauskas, R. J. (1999) Wiley-VCH, New York), section 3.1.1; and Chen et al. J. Am. Chem. Soc. 104:7294-7299 (1982)).

$$\begin{matrix}E = \frac{ln [ 1 - c ( 1 + {ee}_{p} ) ]}{ln [ 1 - c ( 1 - {ee}_{p} ) ]} & eq . ( 1.1 ) \\ E = \frac{ln [ ( 1 - c ) ( 1 - {ee}_{s} ) ]}{ln [ ( 1 - c ) ( 1 + {ee}_{s} ) ]} & eq . ( 1.2 ) \\ E = \frac{ln ( \frac{( 1 - {ee}_{p} )}{1 + ( {ee}_{s} / {ee}_{p} )} )}{ln ( \frac{( 1 + {ee}_{s} )}{1 + ( {ee}_{s} / {ee}_{p} )} )} & eq . ( 1.3 )\end{matrix}$$

As used herein the term “enantiomeric excess” or the symbol “ee,” includes the amount or excess of one enantiomer (whether (R)- or (S)-type) relative to the other in a racemic mixture of such enantiomers. The percent enantiomeric excess is quantitatively expressed by the equation (2):

$$ee = ( \frac{R - S}{R + S} ) 100 \qquad eq . ( 2 )$$

wherein, R is the molar concentration of the (R)-type enantiomer, and S is the molar concentration of the (S)-type enantiomer.

The calculation of ee and enantioselectivity as it relates to enzymatic resolutions of racemic mixtures of chiral compound is further described in U.S. Pat. No. 5,541,080, and U.S. Publication No. 2005/0026260 which are incorporated herein by reference for all purposes.

As used herein, “ATCC” includes the American Type Culture Collection international depository located at 10801 University Blvd. Manassas, Va. 20110-2209 USA. The “ATCC No.” is the accession number of cultures on deposit with the ATCC.

As used herein the term “derivatives and mutants, thereof” used in reference to the description of the microorganisms Acinetobacter baumannii ATCC PTA-8303 or Cryptococcus albidus ATCC PTA-8302 includes microorganisms that essentially correspond to the microorganisms having the aforementioned ATCC numbers. The term “essentially correspond” refers to variations that occur in nature and to artificial variations of the microorganisms that exhibit enantioselective EH activity. In particular, the variations relate to variations in the EH and functional fragments thereof that can be isolated from the microorganisms and to variations in the genome of the organisms encoding EH and functional fragments thereof.

As used herein, the term “reaction medium” includes a solution in which an enzyme catalyst (e.g., EH) and at least one substrate (e.g., rac-EEB) are combined (by for example, dissolving, mixing, suspending). The medium may be aqueous, which is preferably buffered, and may contain solvents in addition to or in lieu of water. The amount of solvent in the reaction medium may be from about none to about 100% of the reaction medium.

Ethyl-3,4-epoxybutyrate is abbreviated as EEB.

(R)-ethyl-3,4-epoxybutyrate is abbreviated as (R)-EEB.

(S)-ethyl-3,4-epoxybutyrate is abbreviated as (S)-EEB.

(R)-ethyl-3,4-dihydroxybutanoate is abbreviated as (R)-diol.

(S)-ethyl-3,4-dihydroxybutanoate is abbreviated as (S)-diol.

Epoxide hydrolase is abbreviated as EH.

### I. Compositions

The present invention relies on the discovery of two novel microorganisms each having epoxide hydrolase (EH) activity. The EHs from these microorganisms can selectively hydrolyze, via epoxide opening, one enantiomer of an alkyl 3,4-epoxybutyrate, preferably ethyl-3,4-epoxybutanoate (EEB), over the other enantiomer.

1. Acinetobacter baumannii ATCC PTA-8303

The inventor of this application has discovered a bacterial microorganism, Acinetobacter baumannii ATCC PTA-8303, that produces an EH which can selectively react with (and hydrolyze) the (S)-enantiomer of EEB to form (S)-ethyl-3,4-dihydroxybutanoate (i.e., (S)-diol) over the (R)-enantiomer in a racemic mixture of EEB (i.e., rac-EEB) (see, Scheme 1A).

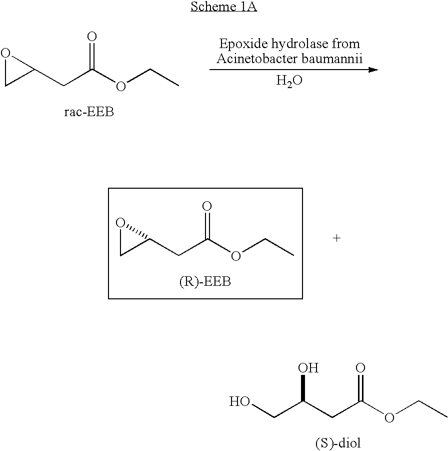

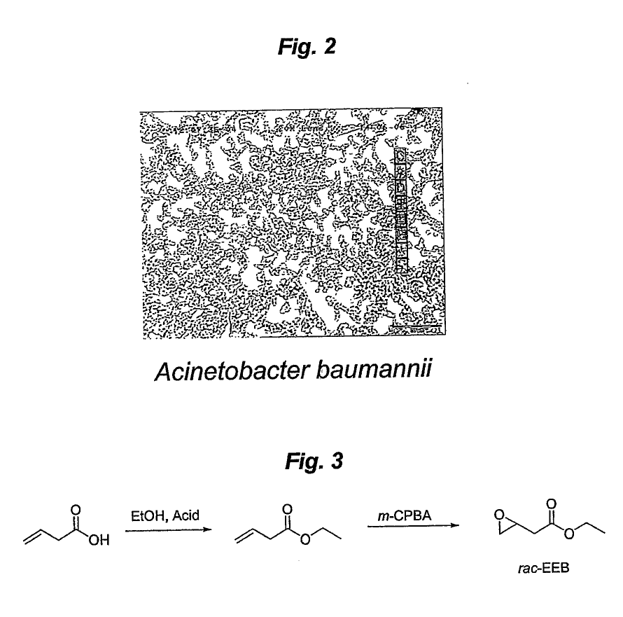

Thus, in one aspect, the present invention provides for a biologically pure culture of Acinetobacter baumannii ATCC PTA-8303 and derivatives and mutants thereof. The microscopic image of Acinetobacter baumannii ATCC PTA-8303 is shown in FIG. 2. The biologically pure culture of Acinetobacter baumannii produces an EH upon aerobic cultivation in an aqueous nutrient medium, preferably containing assimilable sources of carbon, nitrogen and inorganic substances. The EH produced by a biologically pure culture of the microorganism Acinetobacter baumanni ATCC PTA-8303 preferentially hydrolyzes (S)-EEB in a racemic mixture of EEB to form the (S)-diol.

The microbial characteristics of Acinetobacter baumannii ATCC PTA-8303 is summarized in Table 1A below.

TABLE 1A
Microbial characteristics of Acinetobacter baumannii

| | |
|---|---|
| Morphology | Short rod |
| Length | 1.0~1.4 μm |
| Width | 0.4~0.6 μm |
| Gram stain | − |
| Indole production | − |
| Catalase | + |
| Urease | + |
| Oxidase | − |
| Arginine dihydrolase | − |
| Nitrate reduction | − |
| O—F test | oxidative |
| β-glucosidase | − |
| Protease | + |
| β-galactosidase | − |
| L-arabinose | − |
| D-mannose | − |
| D-manitol | − |
| N-acetyl-glucosamine | − |
| D-maltose | − |
| Potassium gluconate | + |
| Capric acid | + |
| Malic acid | + |
| Phenylacetic acid | + |
| Trisodium citrate | + |
| Adipic acid | + |

2. Cryptococcus albidus ATCC PTA-8302

Also, the inventor of this application has discovered a yeast microorganism, Cryptococcus albidus ATCC PTA-8302 that produces an EH which can selectively reacts with the (R)-enantiomer of EEB over the (S)-enantiomer in a racemic mixture of EEB (see, Scheme 1B).

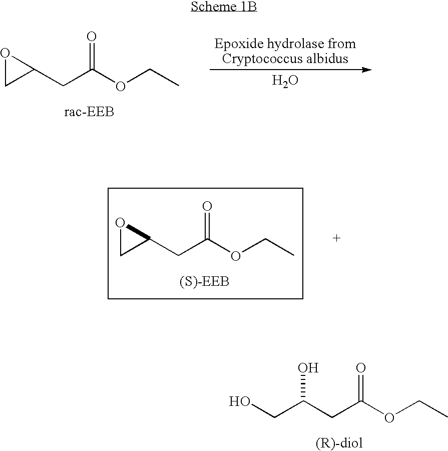

Thus, in another aspect, the present invention provides for a biologically pure culture of Cryptococcus albidus ATCC PTA-8302 and derivatives and mutants thereof. The biologically pure culture of Cryptococcus albidus produces an EH upon aerobic cultivation in an aqueous nutrient medium, preferably containing assimilable sources of carbon, nitrogen and inorganic substances. The EH produced by the biologically pure culture of Cryptococcus albidus preferentially hydrolyzes (R)-EEB in a racemic mixture of EEB to form the (R)-diol.

The microbial characteristics of Cryptococcus albidus ATCC PTA-8302 is summarized in Table 1B below.

TABLE 1B
Microbial characteristics of Cryptococcus albidus

| | |
|---|---|
| Morphology | Oval |
| Length | 3.5~5.0 μm |
| Width | 0.8~1.2 μm |
| Gram stain | + |
| D-Glucose | + |
| Glycerol | − |
| Calcium 2-ketogluconate | + |
| L-Arabinose | − |
| D-Xylose | − |
| Adonitol | − |
| Xylitol | − |
| D-Galactose | − |
| Inositol | − |
| D-Sorbitol | − |
| D-Saccharose | + |
| Methyl-α-D-glucopyranoside | + |
| D-Cellobiose | + |
| N-acetyl-glucosamine | − |
| D-maltose | + |
| D-Lactose | + |
| D-Trehalose | + |
| D-Melezitose | + |
| D-Raffinose | − |

3. Microorganism Culturing:

The microorganisms Acinetobacter baumanni ATCC PTA-8303 or Cryptococcus albidus ATCC PTA-8302 comprising EH activity, can be cultured and grown in a suitable basel medium. There are no particular restrictions on the medium used for this purpose provided that it is allows the microorganisms to grow. This medium is usually an aqueous nutrient medium containing ordinary assimilable sources of carbon, nitrogen and inorganic substances as necessary.

For example, any carbon source may be used provided that the microorganism can utilize it. Specific examples of the carbon source that can be used include sugars such as glucose, fructose, maltose and amylose, alcohols such as sorbitol, ethanol and glycerol, organic acids such as fumaric acid, citric acid, acetic acid and propionic acid and their salts, hydrocarbons such as paraffin as well as mixtures thereof.

Examples of nitrogen sources that can be used include ammonium salts of inorganic salts such as ammonium sulfate and ammonium chloride, ammonium salts of organic acids such as ammonium fumarate and ammonium citrate, nitrates such as sodium nitrate and potassium nitrate, organic nitrogen compounds such as peptones, yeast extract, meat extract and corn steep liquor as well as mixtures thereof.

In addition, ordinary nutrient sources used in media, such as inorganic salts, trace metal salts and vitamins, can also be suitably mixed and used.

There are no particular restrictions on culturing conditions, and culturing can be carried out, for example, for about 12 to about 48 hours while properly controlling the pH and temperature to a pH range of 5 to 8 and a temperature range of 15 to 40° C., respectively, under aerobic conditions. The desired pH can be controlled by the use of any buffer, such as KH₂PO₄ buffer, morpholinoethanesulfonic acid (MES) and the like, or by the addition of nutrient materials which can inherently possess buffering properties.

After growth of the microorganisms is complete, the cells are harvested by conventional methods, e.g., centrifugation and filtration, and then washed and resuspended in the appropriate buffer.

4. Epoxide Hydrolase Purification

Epoxide Hydrolase (EH) can be isolated and purified from the microorganisms Acinetobacter baumanni ATCC PTA-8303 or Cryptococcus albidus ATCC PTA-8302 by the method explained herein. First, a microbial cell extract is prepared from the cells of Acinetobacter baumanni ATCC PTA-8303 or Cryptococcus albidus ATCC PTA-8302 by disrupting the cells using a physical method, such as ultrasonic crushing or an enzymatic method using a cell wall-dissolving enzyme and removing the insoluble fraction by centrifugal separation and so forth.

The EH can then be purified from the cell extract obtained in the above manner by combining ordinary protein purification methods such as anion exchange chromatography, cation exchange chromatography or gel filtration chromatography.

An example of a carrier for use in anion exchange chromatography is Q-Sepharose HP (manufactured by Amersham). The EH is recovered in the non-adsorbed fraction under conditions of pH 8.5 when the cell extract containing the EH is allowed to pass through a column packed with the carrier.

An example of a carrier for use in cation exchange chromatography is MonoS HR (manufactured by Amersham). After adsorbing the EH onto the carrier (in the column) by allowing the cell extract containing the EH to pass through a column packed with the carrier and then washing the column, the EH is eluted with a buffer solution having a high salt concentration. At that time, the salt concentration may be sequentially increased or gradiently increased. For example, in the case of using MonoS HR, the EH adsorbed onto the carrier is eluted at an NaCl concentration of about 0.2 to about 0.5 M.

The EH purified in the manner described above can then be further homogeneously purified by gel filtration chromatography and so forth. An example of the carrier for use in gel filtration chromatography is Sephadex 200 pg (manufactured by Amersham).

The fraction that contains the EH (in the aforementioned purification procedure) can be confirmed by assaying enzyme activity of each fraction according to the kinetic resolution method described later.

### II. Methods

The microorganisms Acinetobacter baumanni ATCC PTA-8303 and Cryptococcus albidus ATCC PTA-8302 each produces an EH having complementary activity and can be employed as versatile biocatalysts for the industrial scale kinetic resolution of racemic EEB, by selective degradation, via epoxide opening (hydrolysis), of one enantiomer of EEB (e.g., (R)-enantiomer) to result in the accumulation of the other enantiomer (e.g.,(S)-enantiomer).

Therefore, in another aspect, the present invention provides for a process of resolving a racemic mixture of EEB comprising: culturing the microorganism Acinetobacter baumanni ATCC PTA-8303 or Cryptococcus albidus ATCC PTA-8302, which is characterized by its ability to preferentially hydrolyze (S)-EEB or (R)-EEB, respectively, in the presence of the racemic mixture to form the (S)-diol or (R)-diol, respectively, in an aqueous nutrient medium under aerobic conditions.

The EHs that are produced by the microorganism Acinetobacter baumanni ATCC PTA-8303 or Cryptococcus albidus ATCC PTA-8302 are responsible for catalyzing the kinetic resolution process, described above, and the EHs from these microorganisms can be used in a method for preparing the (R)- or (S)-enantiomer of EEB in enantioenriched form from racemic EEB. The EHs from these microorganisms can be used in any form, including in whole cells, cell extracts, as partially purified enzymes, purified enzymes, or a mixture thereof.

Therefore, in yet another aspect, the present invention provides for process for preparing (R)-EEB or (S)-EEB comprising: (i) incubating a racemic mixture of EBB with an EH in a form selected from the group consisting of whole cells, cell extracts, partially purified enzyme, purified enzyme, and a mixture thereof, wherein the enzyme and the cell comprising the enzyme is from the microorganism Acinetobacter baumanni ATCC PTA-8303 or Cryptococcus albidus ATCC PTA-8302 respectively, and is characterized by its ability to preferentially hydrolyze (S)-EEB or (R)-EEB, respectively, in the mixture to form the (S)-diol or (R)-diol, respectively, and result in the accumulation of (R)-EEB or (S)-EEB, respectively, in the reaction medium; and (ii) isolating (R)-EEB or (S)-EEB, respectively, from the reaction medium. In one embodiment, the process is carried out in a growing culture of the microorganisms, Acinetobacter baumanni ATCC PTA-8303 or Cryptococcus albidus ATCC PTA-8302, in an aqueous nutrient medium, preferably under aerobic conditions. In other embodiments, the process is carried out using whole cells (e.g., resting cells) of the Acinetobacter baumanni ATCC PTA-8303 or Cryptococcus albidus ATCC PTA-8302 microorganisms, preferably in an aqueous buffer. The whole cells of the microorganism can be prepared as wet cell pellets or as a lyophilized powder. In yet another embodiment, the process is carried out in a microorganism-free reaction medium containing purified or partially purified EH isolated from the microorganism.

In a related aspect, the present invention also provides for a method for producing microbial epoxide hydrolase (EH) comprising the steps of (i) aerobically cultivating the microorganism Acinetobacter baumanni ATCC PTA-8303 or Cryptococcus albidus ATCC PTA-8302 in a nutrient medium containing assimilable sources of carbon, nitrogen and inorganic minerals under conditions suitable for the formation of the epoxide hydrolase; (ii) incubating the culture for a time sufficient to produce the epoxide hydroase; and (iii) recovering the epoxide hydrolase from the nutrient culture medium.

In another related aspect, the present invention provides for the use of an epoxide hydrolase in a form selected from the group consisting of whole cells, cell extracts, partially purified enzyme, purified enzyme, and a mixture thereof, wherein the enzyme is derived from the microorganism Acinetobacter baumannii ATCC PTA-8303 or Cryptococcus albidus ATCC PTA-8302 for preparing (R)-ethyl-3,4-epoxybutyrate or (S)-ethyl-3,4-epoxybutyrate, respectively, from a racemic mixture of ethyl-3,4-epoxybutyrate.

The present discovery allows synthetic access to either the (R)- or the (S)-enantiomer of EEB via a microbial kinetic resolution process catalyzed by Acinetobacter baumannii or Cryptococcus albidus, respectively. The present microbial kinetic resolution process for preparing enantioenriched EEB has many advantages of the processes currently known. For example, the inventive process uses racemic EEB as the starting material which can be synthesized cheaply from inexpensive bulk reagents such as vinylacetic acid, alcohol and m-CPBA. (See, FIG. 3). Moreover, the kinetic resolution process described in the present invention can be carried out under mild reaction conditions (e.g., at ambient temperature) which can reduce the chance that the enantioenriched product can decompose with to reduce the yield.

The enantioenriched products produced by the kinetic resolution process disclosed herein are useful as synthetic intermediates for compounds of high value in the pharmaceutical, agricultural, and fine chemicals industry.

### EXAMPLES

The following examples are provided to illustrate the present invention, and should not be construed as limiting the scope of the invention in any way. The full scope of the invention is defined by the claims presented herein.

### Example 1

The following example describes the general research strategy for discovering of EH activity from microorganisms.

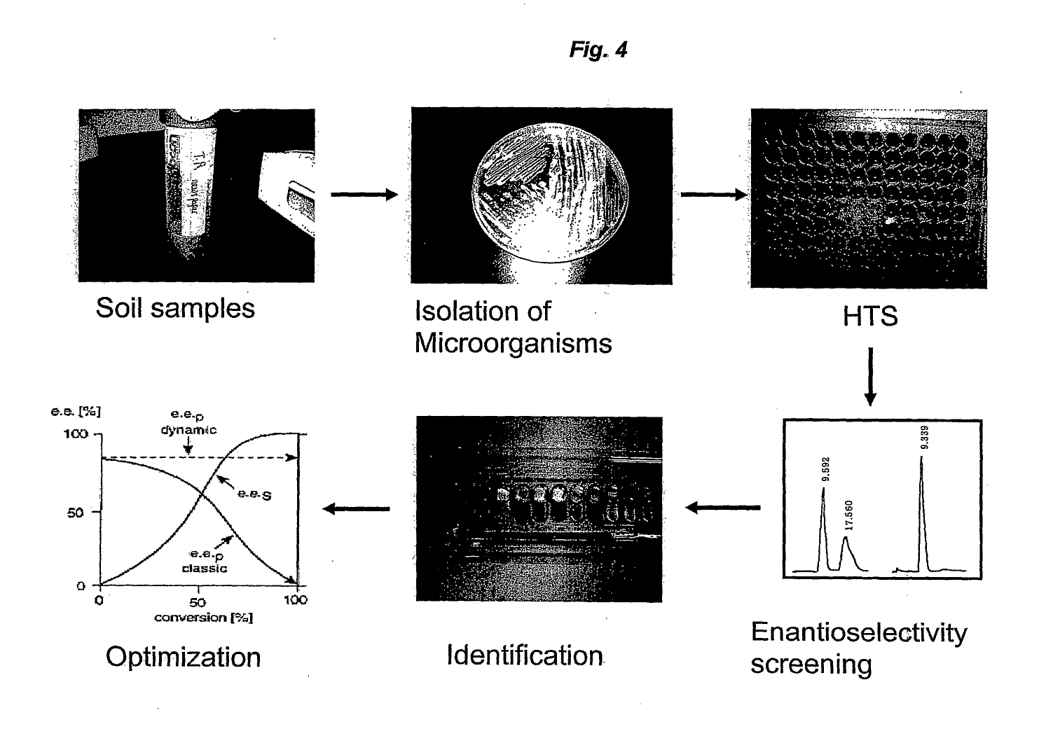

Acinetobacter baumannii ATCC PTA-8303 or Cryptococcus albidus ATCC PTA-8302 were discovered in a research strategy which involved: 1) isolation of microorganisms from the natural environment (e.g. soil samples) which may contain EH activity; 2) high throughput (HTS) screening to identify microorganisms with EH activity and enantioselective screening of microorganisms with EH activity; 3) identification and characterization of newly isolated, pure, microorganisms with EH activity; and 4) optimization of EH activity in the newly isolated, pure, microorganisms for kinetic resolution purposes (see, FIG. 4).

1. Isolation of Microorganisms from Environmental Sources

The soil samples are collected from versatile natural environments, followed by inoculation into the culture medium containing basal nutrients. After preliminary cultivation, cells are cultivated again on the solid medium to be isolated as a single colony.

2. HTS Screening and Enantioselective Screening for Microorganisms with EH Activity

Alkenes (e.g., hexene, heptene, octene or hexadecene) are converted to an epoxide by a monooxygenase and then degraded to vicinal dial, aldehyde or ketone, via epoxide hydrolase-catalyzed hydrolysis or by rearrangement of the epoxide catalyzed by an epoxide isomerase (see, Tetrahedron: Asymmetry, 8, 65 (1997)). Thus, the strains isolated on aliphatic alkenes as a sole carbon source might have epoxide hydrolase activity and be useful for the enantioselective hydrolysis of racemic epoxides. Various microorganisms from soil samples were tested for the utilization of alkene as a sole carbon source. The positive microorganisms obtained are cultivated again and tested for their enantioselectivity by Chiral GC or HPLC using EEB as substrate.

3. Microorganism Identification

The newly isolated microorganisms containing EH activity were identified based on their morphology, Gram staining test, physiological and biological tests including assimilation of carbon and nitrogen sources, detection of catalase activity and fermentation experiment.

4. Kinetic Resolution of EEB by Newly Isolated Microorganisms

The selected microorganisms can be used to catalyze the kinetic resolution of racemic EEB. The selected microorganisms can be employed as a biocatalyst in the form of whole cells and/or cell extracts. Alternatively, EH isolated from the selected microorganisms (e.g., in the form of partially purified enzyme or purified enzyme) can also be employed as catalyst. Any mixture of whole cell, cell extracts, partially purified enzyme, or purified enzyme from the selected microorganisms can also be used as biocatalyst. For industrial applications, it is desirable that the biocatalyst be efficient and stable. To increase EH content, and EH activity from the positive microorganisms, optimization of both culture medium and growth condition is performed wherein each nutritional component and their optimal concentration are to be determined, followed by controlling cell growth, achieve the highest amount of biocatalyst with high EH specific activity.

### Example 2

The specific microorganisms, Acinetobacter baumannii ATCC PTA-8303 and Cryptococcus albidus ATCC PTA-8302, comprising EH activity, were discovered utilizing the specific research strategy discussed below. Its use in the preparation of enantioenriched EEB is also described in the following example.

1. Isolation of Microorganisms from Environmental Sources

The microorganisms originated from soil samples were grown at 30° C. in nutrient broth (pH 7.0), consisting of bacto peptone 10 g/l, yeast extract 5 g/l, and NaCl 10 g/l. After several subcultivations in the medium containing 2% (v/v) aliphatic alkene, such as hexene, heptene, octene or hexadecene, as sole carbon sources, repeated streaking on agar plates containing the same medium was carried out. The medium composition was shown in Table 2.

TABLE 2
The medium composition for screening of alkene-utilizing strains.

| compounds | concentration | |
|---|---|---|
| Alkenes (octene or hexadecene) | 2% | (v/v) |
| Yeast extract | 0.1 | g/l |
| MgCl₂•6H₂O | 0.075 | g/l |
| K₂HPO₄ | 1.55 | g/l |
| NaH₂PO₄ | 0.85 | g/l |
| NH₄Cl | 2.0 | g/l |
| (NH₄)₂SO₄ | 0.1 | g/l |
| Trace element solution^{a} | 5 | ml/l |
| Bacto-Agar | 15 | g/l |

^{a}Trace element solution (CaCl₂ 530 mg/l, FeSO₄•7H₂O 200 mg/l, ZnSO₄•7H₂O 10 mg/l, H₃BO₃ 10 mg/l, CoCl₂•6H₂O 10 mg/l, MnSO₄•5H₂O 4 mg/l, Na₂MoO₄•2H₂O 3 mg/l, NiCl₂•6H₂O 2 mg/l, MgSO₄•7H₂O 200 mg/l)

2. HTS Screening and Enantioselective Screening for Microorganisms with EH Activity

Alkene-utilizing strains grown in nutrient broth were suspended in 2 ml of 100 mM KH₂PO₄ buffer (pH 7.0) supplemented with 10 mM EEB (Jülich Fine Chemical, Germany) in 5 ml screw-cap bottles sealed with a rubber septum and stirred with a magnetic stirrer at 30° C. for 6 h. The reaction mixture was extracted with equal volumes of ethyl acetate, centrifuged for 10 min at 10,000 g, 4° C. and the organic layer was analyzed by gas chromatography (GC). Chiral GC was performed on a Hewlett-Packard 6890 series system equipped with a flame ionization detector. Samples were injected (1 μl) with He as carrier gas and the temperature of injector and detector was 200° C. and 250° C., respectively. The enantiomeric purities of EEB were determined by using a fused silica cyclodextrin capillary β-DEX 225 column (30 m length, 0.25 mm ID, and 0.25 μm film thickness, Supelco Inc.) under temperature gradient condition (column, 80˜150° C. at the rate of 3° C./min). The commercially available (R)- or (S)-EEB was employed as a standard (Jülich Fine Chemical, Germany). Epoxide hydrolase and its enantioselectivity in alkene-utilizing strains were screened with EEB as a substrate. Enantioselective degradation of EEB was observed in case of unidentified strains L8G1B and LL11A5. L8G1B showed (S)-specific degradation activity whereas LL11A5 hydrolyzed (R)-EEB preferentially.

3. Microorganism Identification

The newly isolated microorganisms, L8G1B and LL11A5, containing EH activity were identified based on their morphology, Gram staining test, physiological and biological tests including assimilation of carbon and nitrogen sources, detection of catalase activity and fermentation experiment. L8G1B was identified as Acinetobacter baumannii using API 20NE kit and apiweb (bioMérieux, France) with characteristic features shown in Table 1A (above). LL11A5 was classified as yeast strain based on its shape, size, Gram test, and assimilation pattern. It showed Gram positive result with narrow-necked budding and mucoid colonies. By using API 20 C AUX and apiweb (bioMérieux, France) it was identified as Cryptococcus albidus with assimilating results for various carbon sources shown in Table 1B.

4a. Kinetic Resolution to Prepare (R)-EEB

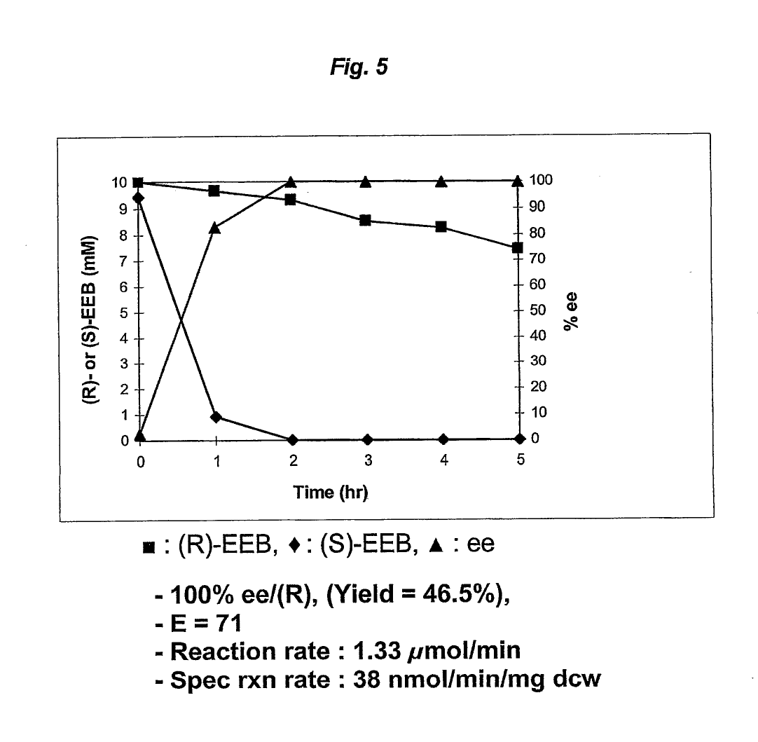

Acinetobacter baumannii was cultured at 30° C. on a medium containing 8.5 g glucose/l, 2 g yeast extract/l, 0.85 g NaH₂PO₄.H₂O/l, 1.55 g K₂HPO₄/l, and 10 ml trace element solution/l for 24 h. Trace element solution is composed of EDTA 1.0 g/l, CaCl₂.2H₂O 0.1 g/l, FeSO₄.7H₂O 0.5 g/l, ZnSO₄.7H₂O 0.2 g/l, CuSO₄.5H₂O 0.02 g/l, CoCl₂.6H₂O 0.04 g/l, MnCl₂.4H₂O 0.1 g/l, Na₂MoO₄.2H₂O 0.02 g/l, MgCl₂.6H₂O 10 g/l, and (NH₄)₂SO₄ 200 g/l. The cells were harvested by centrifugation and washed twice with distilled water. The wet cell pellets (350 mg dry cell weight) were suspended in 10 ml of 100 mM KH₂PO₄ buffer (pH 7.0) in 50 ml screw-cap bottles. The kinetic resolution was initiated by adding racemic EEB at final concentration of 20 mM as substrates. The reaction was carried out at 30° C., 250 rpm in shaking incubator. The kinetic resolution process was monitored by taking samples periodically and terminated when the ee value of residual epoxide reached 100%. Samples (0.2 ml) were withdrawn from the reaction mixture, followed by extraction with ethyl acetate (0.2 ml) and absolute configurations of remaining epoxide in organic phase were determined by chiral GC as described in paragraph 2 of this example. Yield is expressed as a percentage ratio calculated as concentration of most abundant enantiomer divided by initial concentration of its racemate. The newly isolated whole cells of the bacteria Acinetobacter baumannii ATCC PTA-8303 could hydrolyze EEB enantioselectively, produced (after 2 h) enantiopure (R)-EEB (100% ee, 46% Yield). A graph showing the concentration of (R)- and (S)-EEB present in the reaction mixture during the kinetic resolution process as a function of time is found in FIG. 5. The enantiomeric ratio value (E) for this reaction is 71. The formed diol was analysed by chiral HPLC via derivatization with 1-tert-butyldiphenylsilyl chloride (Chiralcel OD, 99.2:0.8 (hexane:EtOH), 1 ml/min, 230 nm).

4b. Kinetic Resolution to Prepare (S)-EEB

Cryptococcus albidus was cultured at 30° C. on the same medium as described in paragraph 4a of this example. The cells were harvested by centrifugation and washed twice with distilled water. The wet cell pellets (710 mg dry cell weight) were suspended in 10 ml of 100 mM KH₂PO₄ buffer (pH 7.0) in 50 ml screw-cap bottles. The kinetic resolution process was initiated by adding racemic EEB at final concentration of 20 mM as substrates. The reaction was carried out at 30° C., 250 rpm in shaking incubator. The kinetic resolution process was monitored by taking samples periodically and is terminated when the ee value of residual epoxide reached 100%. Samples (0.2 ml) were withdrawn from the reaction mixture periodically, followed by extraction with ethyl acetate (0.2 ml) and absolute configurations of remaining epoxide in organic phase were determined by chiral GC as described in paragraph 2 of this example. Yield is expressed as a percentage ratio calculated as concentration of most abundant enantiomer divided by initial concentration of its racemate. Enantioselective hydrolysis of EEB resulted after 4 h in enantiopure (S)-EBB (100% ee, 26% Yield). The formed diol was analysed by chiral HPLC via derivatization with 1-tert-butyldiphenylsilyl chloride (Chiralcel OD, 99.2:0.8 (hexane:EtOH), 1 ml/rain, 230 nm).

### Example 3

The following example illustrates the kinetic resolution of various racemic alkyl 3,4-epoxybutyrates by Acinetobacter baumannii and Cryptococcus albidus via enantioselective epoxide opening.

Synthesis of Racemic alkyl-3,4-epoxybutyrates:

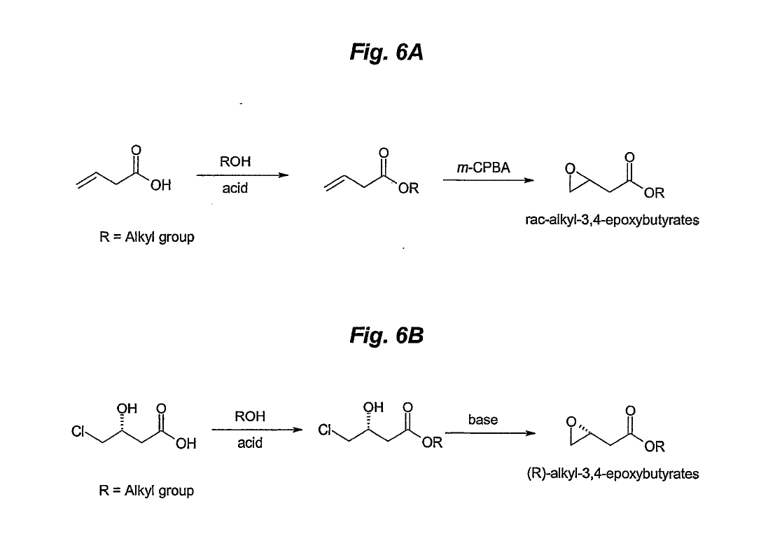

The substrate for kinetic resolution, i.e., alkyl 3,4-epoxybutyrates, were synthesized via acid-catalyzed esterification of 3-butenoic acid, followed by epoxidation with 3-chloroperoxybenzoic acid (m-CPBA) (See, FIG. 6A). Alkyl (R)-3,4-epoxybutyrates were also synthesized via esterification of (R) 4-chloro-3-hydroxybutyric acid with corresponding alcohols, followed by base-catalyzed ring formation and was used as a standard for chiral GC analysis. (See, FIG. 6B)

Kinetic Resolution of alkyl-3,4-epoxybutyrates:

Acinetobacter baumannii and Cryptococcus albidus were cultured at 30° C. on the same medium used in Example 2 paragraph 4a. The cells were harvested by centrifugation and washed twice with distilled water. The wet cell pellets (15 mg dry cell weight for each strain) were suspended in 0.2 ml of 100 mM KH₂PO₄ buffer (pH 7.0) in 1.5 ml screw-cap tubes. The kinetic resolution process was initiated by adding racemic alkyl 3,4-epoxybutyrates at final concentration of 20 mM as substrates. The reaction was carried out at 30° C., 250 rpm in shaking incubator. The reaction was terminated after 3 h of incubation by adding 0.2 ml of ethyl acetate. The remaining epoxides were analyzed chiral GC. The results are presented in Table 3 (below).

GC was performed on Hewlett-Packard 6890 series GC system equipped with FID detector. Organic phase was injected (1 μl) with He as carrier gas and the temperature of injector and detector was 200° C. and 250° C., respectively. The enantiomeric purities of alkyl 3,4-epoxybyturates were determined by using a fused silica cyclodextrin capillary β-DEX 225 column (30 m length, 0.25 mm ID, and 0.25 μm film thickness, Supelco Inc) under temperature gradient conditions (80° C.˜150° C. at the rate of 3° C./min for MEB and EEB, 60° C.˜120° C. at the rate of 1° C./min for BEB, 60° C.˜100° C. at the rate of 1° C./min for TEB, 60° C.˜180° C. at the rate of 1° C./min for OEB and ZEB).

TABLE 3
Enantioselective Hydrolysis of Various Epoxides.

| | Acinetobacter baumannii | | Cryptococcus albidus | |
|---|---|---|---|---|
| Epoxides | ee (%)/abs conf. | Yield (%) | ee (%)/abs conf. | Yield (%) |
| Methyl 3,4-epoxybutyrate (MEB) | 4.3/(R) | 46 | 7.0/(S) | 28 |
| Ethyl 3,4-epoxybutyrate (EEB) | 100/(R) | 46 | 100/(S) | 26 |
| Butyl 3,4-epoxybutyrate (BEB) | 100/(R) | 11 | 5.4/(S) | 48 |
| tert-Butyl 3,4-epoxybutyrate (TEB) | 0 | 45 | 100/(S) | 5 |
| Octyl 3,4-epoxybutyrate (OEB) | 66/(R) | 19 | 2.9/(S) | 6.1 |
| Benzyl 3,4-epoxybutyrate (ZEB) | 10/(R) | 42 | 3.7/(S) | 48 |

### Example 4

The following example illustrates the kinetic resolution of racemic EEB via enantioselective degradation of racemic EEB (via epoxide opening) using lyophilized cell powder of microorganisms.

Acinetobacter baumannii and Cryptococcus albidus stored at −70° C. deep freezer for 3 months were cultured at 30° C. on the same medium used in paragraph 4a of Example 2. The cells were harvested by centrifugation and washed twice with distilled water. The wet cell pellets were frozen for 2 h at −20° C. and then lyophilized for overnight. Dried powders of cells (15 mg) were suspended in 0.2 ml of 100 mM KH₂PO₄ buffer (pH 7.0) in 1.5 ml screw-cap bottles. The kinetic resolution was initiated by adding racemic EEB as substrates at final concentration of 20 mM. The reaction was carried out at 30° C., 250 rpm in shaking incubator. The kinetic resolution was monitored by taking samples periodically and was terminated when the ee value of remaining epoxide reached 100%. The lyophilized cells also showed enantioselective degradation pattern with high yield. Acinetobacter baumannii and Cryptococcus albidus resolved racemic EEB, resulting in (R)- and (S)-EEB with yield of 40% (for 1 h) and 18% (for 3 h), respectively.

Although the foregoing invention has been described in some detail by way of illustration and example for purposes of clarity of understanding, one of skill in the art will appreciate that certain changes and modifications may be practiced within the scope of the appended claims, In addition, each reference provided herein is incorporated by reference in its entirety to the same extent as if each reference was individually incorporated by reference.

1. A biologically pure culture of the microorganism Acinetobacter baumannii of ATCC PTA-8303, and derivatives and mutants thereof.

2. The biologically pure culture according to claim 1, wherein said culture of the microorganism Acinetobacter baumannii produces an epoxide hydrolase upon aerobic cultivation in an aqueous nutrient medium.

3. The biologically pure culture according to claim 2, wherein said aqueous nutrient medium contains assimilable sources of carbon, nitrogen, and inorganic substances.

4. The biologically pure culture according to claim 2, wherein said epoxide hydrolase preferentially hydrolyzes (S)-ethyl-3,4-epoxybutyrate in a racemic mixture of ethyl-3,4-epoxybutyrate to form (S)-ethyl-3,4-dihydroxybutanoate.

5. A process for resolving a racemic mixture of ethyl-3,4-epoxybutyrate comprising culturing the microorganism Acinetobacter baumannii ATCC PTA-8303, which is characterized by its ability to preferentially hydrolyze (S)-ethyl-3,4-epoxybutyrate to form (S)-ethyl-3,4-dihydroxybutanoate, in the presence of said racemic mixture in an aqueous nutrient medium under aerobic conditions.

6. A process for preparing (R)-ethyl-3,4-epoxybutyrate, said process comprising:
(i) incubating a racemic mixture of ethyl-3,4-epoxybutyrate with an epoxide hydrolase in a form selected from the group consisting of whole cells, cell extracts, partially purified enzyme, purified enzyme and a mixture thereof, wherein said enzyme and said cell comprising said enzyme is from the microorganism Acinetobacter baumannii ATCC PTA-8303 and is characterized by its ability to preferentially hydrolyze (S)-ethyl-3,4-epoxybutyrate in said mixture to form (S)-ethyl-3,4-dihydroxybutanoate; and
(ii) isolating (R)-ethyl-3,4-epoxybutyrate from said reaction medium.

7. The process according to claim 6, wherein the process is carried out in a growing culture of the microorganism in an aqueous nutrient medium.

8. The process according to claim 7, wherein said process is performed under aerobic conditions.

9. The process according to claim 6, where said process is carried out in an aqueous buffer using resting cells of the microorganism.

10. The process of claim 6, wherein said process is carried out in a microorganism-free reaction medium containing purified or partially purified epoxide hydrolase enzyme isolated from the microorganism.

11. A biologically pure culture of the microorganism Cryptococcus albidus of ATCC PTA-8302, and derivatives and mutants thereof.

12. The biologically pure culture according to claim 11, wherein said culture of the microorganism Cryptococcus albidus produces an epoxide hydrolase upon aerobic cultivation in an aqueous nutrient medium.

13. The biologically pure culture according to claim 12, wherein said aqueous nutrient medium contains assimilable sources of carbon, nitrogen, and inorganic substances.

14. The biologically pure culture according to claim 12, wherein said produced epoxide hydrolase is capable of preferentially hydrolyzing (R)-ethyl-3,4-epoxybutyrate in a racemic mixture of ethyl-3,4-epoxybutyrate to form (R)-ethyl-3,4-dihydroxybutanoate.

15. A process for resolving a racemic mixture of ethyl-3,4-epoxybutyrate comprising culturing the microorganism Cryptococcus albidus ATCC 8302, which is characterized by its ability to preferentially hydrolyze (R)-ethyl-3,4-epoxybutyrate to form (R)-ethyl-3,4-dihydroxybutanoate, in the presence of said racemic mixture in an aqueous nutrient medium under aerobic conditions.

16. A process for preparing (S)-ethyl-3,4-epoxybutyrate comprising the steps of:
(i) incubating a racemic mixture of ethyl-3,4-epoxybutyrate with an epoxide hydrolase in a form selected from the group consisting of whole cells, cell extracts, partially purified enzyme, purified enzyme and mixtures thereof, wherein said enzyme and said cell comprising said enzyme is from the microorganism Cryptococcus albidus ATCC PTA-8302 and is characterized by its ability to preferentially hydrolyze (R)-ethyl-3,4-epoxybutyrate in said mixture to form (R)-ethyl-3,4-dihydroxybutanoate; and
(ii) isolating (S)-ethyl-3,4-epoxybutyrate from said reaction medium.

17. The process according to claim 16, wherein the process is carried out in a growing culture of the microorganism in an aqueous nutrient medium.

18. The process according to claim 17, where said process is performed under aerobic conditions.

19. The process according to claim 16, wherein said process is carried out in an aqueous buffer using resting cells of said microorganism.

20. The process of claim 16, wherein said process is carried out in a microorganism-free reaction medium containing purified or partially purified epoxide hydrolase isolated from the microorganism.

21. A method for producing the microbial epoxide hydrolase comprising the steps of:
(i) aerobically cultivating the microorganism Acinetobacter baumannii ATCC PTA-8303 under conditions suitable for the formation of the epoxide hydrolase in a nutrient medium containing assimilable sources of carbon, nitrogen, and inorganic minerals;
(ii) incubating the culture for a time sufficient to produce said epoxide hydrolase; and
(iii) recovering said epoxide hydrolase from said nutrient medium.

22. A method for producing the microbial epoxide hydrolase comprising the steps of:
(i) aerobically cultivating the microorganism Cryptococcus albidus ATCC PTA-8302 under conditions suitable for the formation of the epoxide hydrolase in a nutrient medium containing assimilable sources of carbon, nitrogen, and inorganic minerals;
(ii) incubating the culture for a time sufficient to produce said epoxide hydrolase; and
(iii) recovering said epoxide hydrolase from said nutrient medium.

23-24. (canceled)
